(19) United States
(12) Patent Application Publication
(10) Pub. No.: US 2017/0119884 A1
(43) Pub. Date: May 4, 2017

(54) Drug having steroids and fatty compound

(71) Applicants: Ken-Chuan Chou, Kaohsiung (TW); Yai-Min Chen, Kaohsiung (TW)

(72) Inventors: Ken-Chuan Chou, Kaohsiung (TW); Yai-Min Chen, Kaohsiung (TW)

(21) Appl. No.: 15/408,440

(22) Filed: Jan. 18, 2017

(51) Int. Cl. A61K 47/12; A61K 47/10; A61K 47/46; A61K 9/00; A61K 47/06; A61K 47/24; A61K 47/36; A61K 47/32; A61K 31/56; A61K 47/18

(52) U.S. Cl. CPC A61K 47/12; A61K 31/56; A61K 47/10; A61K 47/46; A61K 47/18; A61K 47/06; A61K 47/24; A61K 47/36; A61K 47/32; A61K 9/0014

(57) ABSTRACT

A drug is provided with an emulsifier and additive free fatty compound, and steroids. The fatty compound has a core structure similar to stratum corneum. In one embodiment, the fatty compound includes 75-85 wt % of aqua, 35-45 wt % of caprylic/capric triglyceride, 1-10 wt % of pentylene glycol, 1-9 wt % of hydrogenated lecithin, 1-9 wt % of butyrospermum parkii butter, 1-7 wt % of glycerin, 1-5 wt % of ceramide NP, and 1.2-2.2 wt % of squalane.

### BACKGROUND OF THE INVENTION

1. Field of the Invention

The invention relates to drugs and more particularly to a drug having steroids and a fatty compound.

2. Description of Related Art

Conventional drugs include steroids and a fatty compound having additives including spices, preservatives, colors, mineral oils, silicides, and amines. However, such drugs may have adverse effects such as skin aging and damaging stratum corneum.

Thus, the need for improvement still exists.

### SUMMARY OF THE INVENTION

It is therefore one object of the invention to provide a drug comprising a fatty compound without emulsifier; and steroids.

The above and other objects, features and advantages of the invention will become apparent from the following detailed description taken with the accompanying drawings.

### BRIEF DESCRIPTION OF THE DRAWINGS

FIG. 1 is a table of compositions of a fatty compound of a drug according to a first preferred embodiment of the invention;

FIG. 2 is a table of compositions of a fatty compound of a drug according to a second preferred embodiment of the invention; and

FIG. 3 is a table of compositions of a fatty compound of a drug according to a third preferred embodiment of the invention.

### DETAILED DESCRIPTION OF THE INVENTION

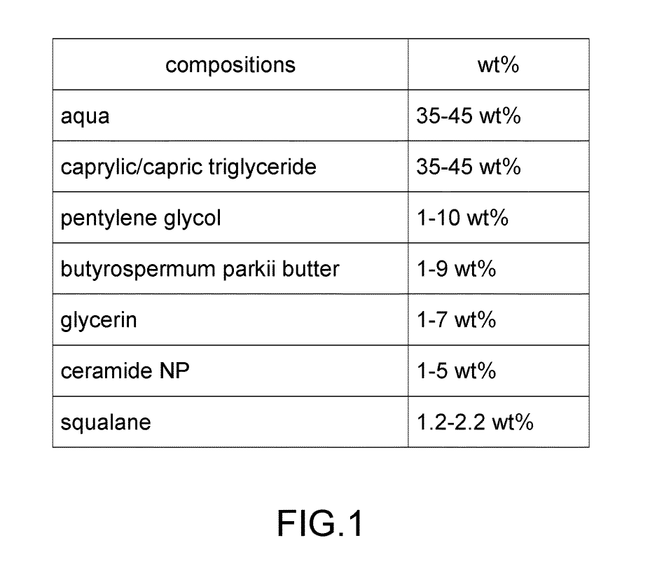

Referring to FIG. 1, it is a table of compositions of a fatty compound of a drug in accordance with a first preferred embodiment of the invention. The fatty compound comprises 75-85 wt % of aqua, 35-45 wt % of caprylic/capric triglyceride, 1-10 wt % of pentylene glycol, 1-9 wt % of butyrospermum parkii butter, 1-7 wt % of glycerin, 1-5 wt % of ceramide NP (natural product), and 1.2-2.2 wt % of squalane. The drug further comprises steroids.

The fatty compound is without emulsifier.

The fatty compound has a core structure similar to stratum corneum.

The fatty compound is without spices, preservatives, colors, mineral oils, silicides, and amines.

It is envisaged by the Invention that the drug can protect stratum corneum and provide sufficient moisture to the skin as well as can heal wound or function as a skin care ointment without adverse effects.

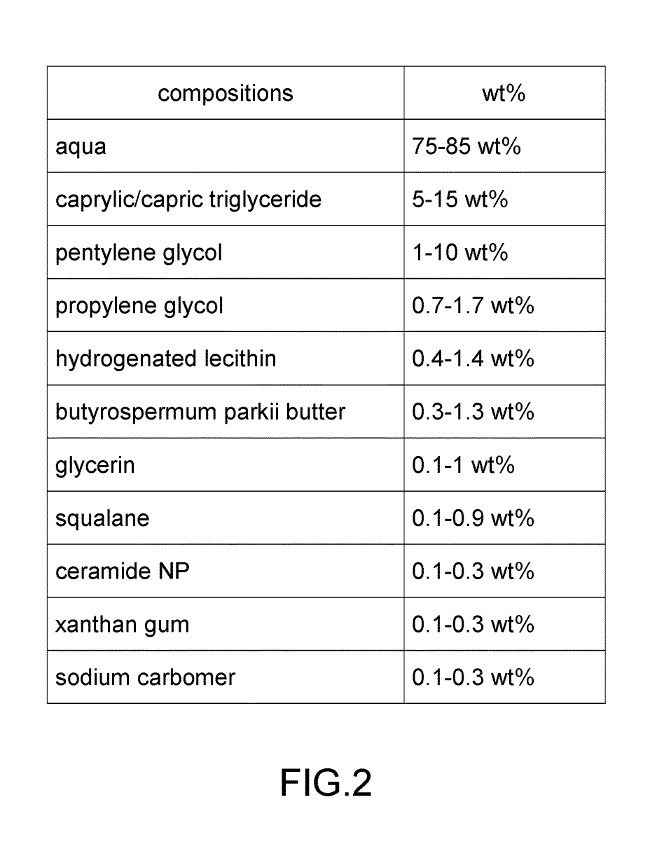

Referring to FIG. 2, it is a table of compositions of a fatty compound of a drug in accordance with a second preferred embodiment of the invention. The fatty compound comprises 75-85 wt % of aqua, 5-15 wt % of caprylic/capric triglyceride, 1-10 wt % of pentylene glycol, 0.7-1.7 wt % of propylene glycol, 0.4-1.4 wt % of hydrogenated lecithin, 0.3-1.3 wt % of butyrospermum parkii butter, 0.1-1 wt % of glycerin, 0.1-0.9 wt % of squalane, 0.1-0.3 wt % of ceramide NP, 0.1-0.3 wt % of xanthan gum, and 0.1-0.3 wt % of sodium carbomer. The drug further comprises steroids.

The fatty compound is without emulsifier.

The fatty compound has a core structure similar to stratum corneum.

The fatty compound is without spices, preservatives, colors, mineral oils, silicides, and amines.

It is envisaged by the Invention that the drug can protect stratum corneum and provide sufficient moisture to the skin as well as can heal wound or function as a skin care ointment without adverse effects.

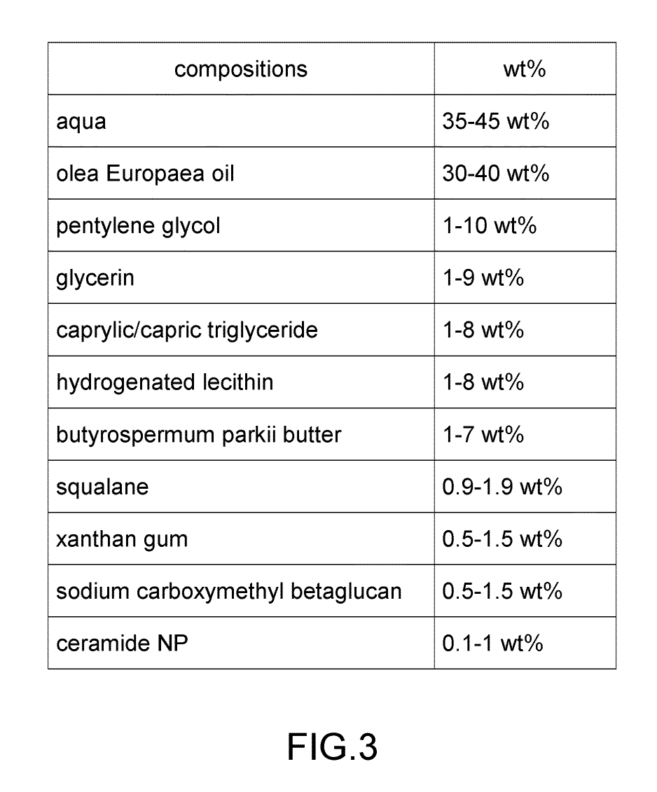

Referring to FIG. 3, it is a table of compositions of a fatty compound of a drug in accordance with a third preferred embodiment of the invention. The fatty compound comprises 35-45 wt % of aqua, 30-40 wt % of olea Europaea oil, 1-10 wt % of pentylene glycol, 1-9 wt % of glycerin, 1-8 wt % of caprylic/capric triglyceride, 1-8 wt % of hydrogenated lecithin, 1-7 wt % of butyrospermum parkii butter, 0.9-1.9 wt % of squalane, 0.5-1.5 wt % of xanthan gum, 0.5-1.5 wt % of sodium carboxymethyl betaglucan, and 0.1-1 wt % of ceramide NP. The drug further comprises steroids.

The fatty compound is without emulsifier.

The fatty compound has a core structure similar to stratum corneum.

The fatty compound is without spices, preservatives, colors, mineral oils, silicides, and amines.

It is envisaged by the Invention that the drug can protect stratum corneum and provide sufficient moisture to the skin as well as can heal wound or function as a skin care ointment without adverse effects.

While the invention has been described in terms of preferred embodiments, those skilled in the art will recognize that the invention can be practiced with modifications within the spirit and scope of the appended claims.

What is claimed is:

1. A drug comprising:
an emulsifier free fatty compound; and
steroids.

2. The drug of claim 1, wherein the fatty compound has a core structure similar to stratum corneum.

3. The drug of claim 2, wherein the fatty compound comprises aqua, caprylic/capric triglyceride, pentylene glycol, butyrospermum parkii butter, glycerin, ceramide NP, and squalane.

4. The drug of claim 3, wherein the fatty compound comprises 75-85 wt % of aqua, 35-45 wt % of caprylic/capric triglyceride, 1-10 wt % of pentylene glycol, 1-9 wt % of hydrogenated lecithin, 1-9 wt % of butyrospermum parkii butter, 1-7 wt % of glycerin, 1-5 wt % of ceramide NP, and 1.2-2.2 wt % of squalane.

5. The drug of claim 2, wherein the fatty compound comprises aqua, caprylic/capric triglyceride, pentylene glycol, propylene glycol, hydrogenated lecithin, butyrospermum parkii butter, glycerin, squalane, ceramide NP, xanthan gum, and sodium carbomer.

6. The drug of claim 5, wherein the fatty compound comprises 75-85 wt % of aqua, 5-15 wt % of caprylic/capric triglyceride, 1-10 wt % of pentylene glycol, 0.7-1.7 wt % of propylene glycol, 0.4-1.4 wt % of hydrogenated lecithin, 0.3-1.3 wt % of butyrospermum parkii butter, 0.1-1 wt % of glycerin, 0.1-0.9 wt % of squalane, 0.1-0.3 wt % of ceramide NP, 0.1-0.3 wt % of xanthan gum, and 0.1-0.3 wt % of sodium carbomer.

7. The drug of claim 2, wherein the fatty compound comprises aqua, olea Europaea oil, pentylene glycol, glycerin, caprylic/capric triglyceride, hydrogenated lecithin, butyrospermum parkii butter, squalane, xanthan gum, sodium carboxymethyl betaglucan, and ceramide NP.

8. The drug of claim 7, wherein the fatty compound comprises 35-45 wt % of aqua, 30-40 wt % of olea Europaea oil, 1-10 wt % of pentylene glycol, 1-9 wt % of glycerin, 1-8 wt % of caprylic/capric triglyceride, 1-8 wt % of hydrogenated lecithin, 1-7 wt % of butyrospermum parkii butter, 0.9-1.9 wt % of squalane, 0.5-1.5 wt % of xanthan gum, 0.5-1.5 wt % of sodium carboxymethyl betaglucan, and 0.1-1 wt % of ceramide NP.

9. The drug of claim 2, wherein the fatty compound is without spices, preservatives, colors, mineral oils, silicides, and amines.
